(19) United States
(12) Patent Application Publication
(10) Pub. No.: US 2011/0312089 A1
(43) Pub. Date: Dec. 22, 2011

(54) PROCESS FOR CULTIVATING CELLS

(76) Inventors: Adrian Knight, Cleveland (GB); Bhupendra Vallabh Kara, Cleveland (GB); Rachel Yvonne Richer, Cleveland (GB)

(21) Appl. No.: 13/057,382

(22) PCT Filed: Aug. 6, 2009
(86) PCT No.: PCT/GB2009/001938

§ 371 Date: Apr. 29, 2011

(30) Foreign Application Priority Data
Aug. 7, 2008 (GB) 0814443.8

(51) Int. Cl. C12N 5/077; C12N 5/071

(52) U.S. Cl. 435/366

(57) ABSTRACT

A process for cultivation of differentiated human cells retaining stem cell potential is provided. The process comprises culturing differentiated human cells retaining stem cell potential anchored to a microcarrier selected from the group consisting of gelatin microcarriers and quaternary ammonium derivatised polystyrene microcarriers.

The present invention concerns a process for cultivating differentiated human cells retaining stem cell potential.

Stem cells and cells having stem cell potential are of increasing interest in many therapeutic areas. Such cells are typically produced by cultivation of cells anchored to a solid support medium. Mouse embryonic stem cells have been shown to grow in stirred suspension culture on Sigma SoloHill (Fibronectin-coated polystyrene) and Cytodex 3 (collagen) microcarriers. (Fok E Y et al, Stem Cells 2005, 23(9): 1333-42). Porcine bone marrow-derived primary mesenchymal stem cells have been shown to grow on Cytodex type 1, 2 and 3 (collagen) microcarriers (Frauschuh S et al, Biotechnol Prog. 2007, 23(1): 187-193). Embryonic feline lung fibroblasts have been shown to grow on Cytodex 1 in wave and stirred tank bioreactors (Hundt B et al, Vaccine 2007, 25(10): 3987-95). Mouse embryonic stem cells have also been shown to grow on Cytodex 3 microcarriers in spinner flasks (Abranches E et al, Biotechnol Bioeng. 2007, 96(6): 1211-21). During the studies resulting in the present invention, it was discovered that such microcarriers suffer from a number of deficiencies rendering them unsuitable for reliable and scaleable production of differentiated human cells retaining stem cell potential.

US2007/0264713 discloses that many different microcarriers can be employed to grow many different types of stem cells. A gelatin microcarrier is exemplified along with a collagen microcarrier in the cultivation of human embryonic stem cells. The results show that the collagen microcarrier gives an increase in cells three times greater then the gelatin microcarrier.

According to one aspect of the present invention, there is provided a process for cultivation of differentiated human cells retaining stem cell potential which comprises culturing differentiated human cells retaining stem cell potential anchored to a microcarrier selected from the group consisting of gelatin microcarriers and quaternary ammonium derivatised polystyrene microcarriers.

The cells are anchored to the microcarrier by methods known in the art, for example including by attachment to the surface of the microcarrier, by attachment to the internal structure of macroporous microcarriers, or by physical entrapment inside the internal structure of macroporous microcarriers.

Gelatin microcarriers which can be employed in the method of the present invention can be composed of gelatin particles, cross linked gelatin particles or gelatin used as a coating on carrier materials, for example polystyrene or glass particles. The gelatin can be of natural source or recombinantly or synthetically produced. Gelatins or collagens can be crosslinked via the amine groups of lysine, via carboxyl groups glutamic acid or aspartic acid, or a combination thereof. Gelatin microcarriers are typically approximately spherical but can have other shapes and can be either porous or solid. Both porous and solid types of microcarriers are commercially available. Microcarriers may have a dense surface with dents to facilitate anchorage of the cells. Macroporous gelatin microcarriers for example are available commercially from Percell Biolytica AB, Sweden under the tradename Cultispher, especially Cultispher G and Cultispher S. Gelatin macroporous microcarriers are typically comprise particles based on a highly cross linked gelatin matrix, often with a particle size of from 10-500 μm and form a polymer matrix enclosing a large number of cavities typically having a diameter of from 1-50 μm. The particle size range of the microcarrier is commonly selected to be large enough to accommodate the anchorage of the stem cell whilst being small enough to form suspensions with properties suitable for use in cell culture bioreactors such as shake flasks, roller bottles, spinner flasks, wave bioreactors and stirred tank bioreactor systems.

Quaternary ammonium derivatised polystyrene microcarriers which can be employed in the present invention comprise and amino-group attached to polystyrene, the amino group being quaternised, preferably by three alkyl groups, especially three C₁₋₄ alkyl groups. Examples of such microcarriers include HyQSphere™ HLX11-170, commercially available from Hyclone/ThermoFisher Scientific Inc.

Gelatin microcarriers are particularly suited for applications where cells are cultured on different batches of microcarriers, where cells are cultivated on a first batch of microcarrier, and then to expand the scale of cultivation, cells are harvested and then reattached, optionally after storage for a period, for example storage in a freezer, to a microcarrier, typically a larger batch of microcarrier, or to multiple cultivators totaling a larger amount of microcarrier. Such culturing—harvesting and reattachment can be repeated as often as necessary to produce the desired quantity of cells. Gelatin microcarriers can be employed as the initial microcarrier or as subsequent microcarriers, or an alternative microcarrier, especially a quaternary ammonium-derivatised polystyrene microcarrier, can be employed as the initial microcarrier, with subsequent reattachment post harvesting being to a gelatin microcarrier.

Quaternary ammonium-derivatised polystyrene microcarriers are suited to applications where the cells are seeded onto the microcarrier and cultured on that microcarrier until harvest, with no detachment and reattachment, or as the initial microcarrier with subsequent harvesting and reattachment to a gelatin microcarrier. Additionally, a quaternary ammonium-derivatised polystyrene microcarrier may be employed as the final microcarrier, with other microcarriers being employed for earlier cultivation steps.

Differentiated human cells retaining stem cell potential which can be produced by the method of the present invention are known in the art, and are preferably adult cells, especially mesenchymal cells, and most preferably dermal cells, including adiposyte cells. Especially preferred cells include dermal sheath cells, dermal fibroblast cells and dermal papilla cells, especially dermal sheath cells as described in EP-A-0980270, dermal papilla cells as described in U.S. Pat. No. 5,851,831 and most especially dermal fibroplast cells as described in co-pending British patent application no. 0913469.3.

The process according to the present invention can be carried out in vessels known in the art, including tissue culture flasks, shake flasks, spinner flasks, stirred tank bioreactors, disposable bag based bioreactor systems such as wave cell culture systems, and expanded bed bioreactor systems. Options for large scale production also include roller bottles, hollow fibre systems, single, multi-plate or stacked-plate culture systems and cell cubes.

The process of the present invention is commonly carried out by cultivating the cells in a stirred tank culture vessel system. Typically, the cells, microcarriers and nutrient medium are supplied to the culture vessel and stored under conditions conducive to cell propagation. If desired, additional culture medium may be added to the culture vessel system until the culture is finally terminated and cells harvested. The process of the present invention is carried out by cultivating the cells under conditions conducive to the growth of the cells whilst retaining the cell's stem cell potential. Culture conditions, such as temperature, pH, dissolved oxygen (including hypoxic low oxygen conditions) and the like, are those known to be optimal for the particular cell and will be apparent to the skilled person (see, e.g., Animal Cell Culture: A Practical Approach 2^{nd} Ed., Rickwood, D. and Hames, B. D., eds., Oxford University Press, New York (1992)). Typically, cells are cultured at a pH around neutral pH, commonly in the range of from 6.5 to 7.5 and a temperature in the range of from about 30 to 38° C.

In certain aspects of the present invention, the cells are cultured in a vessel which is agitated with a with an agitator comprising two or more impellers, commonly rotational stirrers, located at different depths in the culture vessel. Preferably, the impellers are mounted about a common shaft. Where two impellers are employed, one impeller is preferably located towards the base of the medium in the culture vessel, such as in the lower third of the vessel, with the second impeller located either towards the middle of the medium in the vessel, such as in the middle third, or towards the top of the medium in the vessel, such as in the top third. In many embodiments, each impeller comprises two, three of four blades angled compared with the axis of the impeller shaft(s), such as propeller-type blades. Preferably, where rotational stirrers are employed as impellers, the stirrers are selected to have a stirrer diameter:vessel diameter ratio of at least 0.25:1, such as in the range from 0.3:1 to 0.7:1.

In other aspects of the present invention, the cells are cultured in a vessel which is agitated with a with an agitator comprising at least one helical blade, and preferably a helical stirrer as described in International patent application WO00/66258.

In one embodiment of the present invention the process is operated in one culture vessel, the cells are inoculated directly into the culture vessel containing microcarriers, the cells are propagated until the desired cell density is reached and the cells harvested.

In another embodiment of the present invention the process is operated in at least two distinct cell culture vessels. Cultivation may take place in one or more seed expansion vessels, followed by cultivation in a cell production vessel, and from which the cell product is harvested. The multiple seed expansion process may take in culture vessels of increasing size until a sufficient number of cells is obtained for the inoculation of the final production cell culture vessel. The seed expansion culture vessels can be of the same type (e.g. tissue culture flasks, shake flasks, roller bottles, spinner flasks, wave bioreactors, stirred tank bioreactors) but increasing in size as the seed expansion progresses or can be a mixture of culture systems increasing in size as the seed culture is expanded in readiness for transfer to the production bioreactor (e.g. tissue culture flasks to shake flasks to spinner flasks to stirred tank bioreactor systems, etc).

According to a preferred aspect of the invention, fed batch or continuous cell culture conditions are devised to enhance growth of the cells in culture. Culture conditions, such as temperature, pH, dissolved oxygen (dO₂) and the like, are those used with the particular cell and will be apparent to the ordinarily skilled artisan. Generally, the pH is adjusted to a level between about 6.5 and 7.5 using either an acid (e.g., CO₂) or a base (e.g., Na₂CO₃ or NaOH). A suitable temperature range for culturing cells is often between about 30° to 38° C. and a suitable dO₂ is often between 5-90% of air saturation.

Cells can be released from gelatin microcarriers using procedures known in the art which limit the potential damage to the cell harvest during cell recovery processes. Cells are commonly released from gelatin microcarriers with the aid of a proteolytic enzyme, for instance, collagenase. Subsequent to the termination of the growth of the cells, the cells are released, when desired, from the carrier with such a proteolytic enzyme.

The medium exchange can be performed by allowing the microcarriers to settle to the bottom of the cell culture vessel, after which a selected percentage of the cell culture growth medium volume is removed and a corresponding percentage of fresh cell culture growth medium is added to the cell culture vessel. The microcarriers are then re-suspended in the medium and this process of medium removal and replacement are typically repeated.

Various terms are used to describe cells in culture. ‘Cell culture’ generally refers to cells taken from a living organism and grown under controlled conditions. A primary cell culture is a culture of cells, tissues or organs taken directly from organisms before the first subculture. Cells are expanded in culture when they are placed in a growth medium under conditions that facilitate growth and/or division, resulting in a larger population of cells. A cell line is a population of cells formed by one or more subcultivations of a primary cell culture. Each round of subculturing is referred to as a passage. It will be understood by those skilled in the art that there may be many population doublings during the period of passaging.

Culture media suitable for use in the process of the present invention are known in the art, including, basal media supplemented with serum, serum-free media, protein-free media or chemically defined growth media. A ‘conditioned’ medium may be used. A conditioned medium is one in which a specific cell or population of cells has been cultured and then removed. While these cells are cultured in the medium they secrete cellular factors that can provide support to other cells. The medium containing the cellular factors is the ‘conditioned’ medium. The cells used for conditioning the medium can be of the same type as subsequently cultivated, a different cell type or a combination of both.

The present invention is illustrated without limitation by the following examples.

### Establishment of Cell Lines

Hair follicle mesenchymal cells were isolated essentially as described in EP980270 with the modifications described below. Human skin tissue samples were washed 3 times with Minimal Essential Medium (MEM, Sigma) containing 1 μg/ml amphotericin and 10 μg/ml gentamycin. Under a dissecting microscope, anagen ‘end bulbs’ were dissected using fine surgical scissors and placed into small volumes (typically 100-200 μl) of MEM. The end bulbs were inverted using needles, and the papilla dissected and the sheath extracted. The papillae and sheaths were then transferred separately to 4 well cell culture plates (Nunc). Ten papillae and 10 sheath were transferred per well in 1 ml of MEM supplemented with 20% foetal bovine serum, 0.5 μg/ml amphotericin and 5 μg/ml gentamycin. The four well cell culture plates were incubated under sterile and standard conditions (37° C., 5% carbon dioxide). After 10 days cell growth, cells were detached from each well (using standard methods well established in the art) and transferred separately to a 35 mm diameter cell culture dish (Nunc). When cell growth was confluent, the dermal sheath (hereinafter referred to as ‘AVDS’) and dermal papilla (hereinafter referred to as ‘AVDP’) cell lines were detached as previously indicated and transferred to T25 cell culture flasks (Nunc) for further expansion under the conditions described above.

AVDS and AVDP cell lines were established form a number of different human tissue samples. A summary of these cell lines which are described in the following examples is provided in Table 1 below.

### Establishment of Dermal Fibroblast Cell Lines

Dermal fibroblast (hereinafter referred to as ‘AVDF’) cell lines were established from the same human skin tissue samples described above. The dermis was separated from the adipose layer and then dissected'under a microscope into pieces of approximately 2-3 mm² surface area. Dissected tissue was transferred to a T25 cell culture flask (Nunc) containing MEM supplemented as described for the dermal sheath and dermal papilla cell lines. The T25 cell culture flasks containing dermal fibroblast (AVDF) cell lines were incubated under sterile and standard conditions (as described previously). The dermal fibroblast (AVDF) cell lines were then further expanded using the same conditions when the cultures had reached confluency.

AVDF cell lines were established form a number of different human tissue samples. A summary of these cell lines which are described in the following examples is provided in Table 1 below.

TABLE 1
Summary of Cell Lines

| Cell Line Designation | Type |
|---|---|
| AVDS1 | Dermal sheath |
| AVDP1 | Dermal papilla |
| AVDS2 | Dermal sheath |
| AVDS3 | Dermal sheath |
| AVDS4 | Dermal sheath |
| AVDS5 | Dermal sheath |
| AVDF1 | Dermal fibroblast |
| AVDF2 | Dermal fibroblast |
| AVDS6 | Dermal sheath |
| AVDF3 | Dermal fibroblast |

### COMPARATIVE EXAMPLE 1

Cytodex I and Cytodex III microcarriers (Amersham Biosciences) were prepared using standard preparation and sterilisation conditions described by the manufacturer. Cytodex I and III microcarriers were then used at a concentration of 5 cm²/ml and were seeded with AVDS2 cell line at a cell concentration of 2.2×10⁵ cells per 125 ml cell culture shake flask (Corning) in a total volume of 15 ml of MEM (Sigma M4655) supplemented with 10% fetal bovine serum (FBS). The cell culture flasks were placed (day=‘0’) in an orbital shaker and incubated at 37° C. with agitation at 70 rpm. The MEM growth medium supplemented with 10% FBS was replaced in each flask on day 3. The cells were harvested after 5 days using standard cell detachment methods well established in the art and the number of AVDS viable cells determined using trypan blue staining and cell counts as is well established in the art. Surprisingly, no viable AVDS cells were recovered from the culture with either Cytodex I or Cytodex III microcarriers.

Cytodex I and Cytodex III microcarriers are widely-used successfully in the industry for the culture of a wide range of mammalian cell types. Both Cytodex I and Cytodex III are composed of Dextran. Cytodex I is composed of a dextran matrix with cationic DEAE groups whilst Cytodex III is composed of dextran matrix coated with denatured porcine collagen. Given the differences in the surface chemistry between Cytodex I and Cytodex III and their wide-spread use in industry, including, for example, the use of these microcarriers to grow embryonic stem cells, bone marrow derived mesenchymal stem cells and lung fibroblasts, one skilled in the art would have expected that these microcarriers would also support growth of the dermal sheath cells (AVDS).

### COMPARATIVE EXAMPLE 2

2D MicroHex microcarriers (Nunc) were used to culture cell lines AVDS1, AVDS2 and AVDP1. The microcarriers were prepared and sterilised using standard methods recommended by the manufacturer. The 2D MicroHex microcarriers (Nunc) are composed of the same material/surface chemistry that is used for static cell culture (Example 1 and 2). Microcarrier concentrations of 1.0 cm²/ml, 2.0 cm²/ml and 4.4 cm²/ml were used and 4×10⁵ AVDS1, AVDS2, AVDS3 and AVDP1 cells per 250 ml cell culture shake flask (Corning) were seeded in a 40 ml total volume MEM (Sigma M4655) supplemented with 10% fetal bovine serum (FBS). The cell culture flasks were placed in an orbital shaker at 37° C. with agitation at 70 rpm. The cell culture flasks were harvested after 3 to 10 days and the total viable cell concentration determined using methods well established in the art. The results obtained are presented in Table 2.

The poor growth achieved by mesenchymal dermal stem cell lines AVDS1, AVDS2 and AVDP1 were not expected and totally surprising given that in static culture these cell lines adhere to the surface of the flask and proliferate readily. The 2D MicroHex microcarriers are composed of the same material/surface chemistry as that used in the static cell culture flasks.

TABLE 2
Growth of cell lines AVDS1, AVDS2, AVDS3 and AVDP1 on MicroHex
Microcarriers

| | MicroHex | Time | | |
|---|---|---|---|---|
| | Concentration | in culture | Harvest cell | Harvest viability |
| Cell line | (cm²/ml) | (days) | number | (% viable cells) |
| AVDS1 | 4.4 | 7 | 3.9 × 10⁵ | 79 |
| AVDP1 | 4.4 | 6 | 4.5 × 10⁵ | 63 |
| AVDS2 | 4.4 | 6 | 4.4 × 10⁵ | 48 |
| AVDS2 | 2.0 | 10 | 3.2 × 10⁵ | 72 |
| AVDS2 | 1.0 | 6 | 4.1 × 10⁵ | 53 |
| AVDS3 | 2.0 | 3 | 5.0 × 10⁵ | 76 |

### EXAMPLE 3

The following microcarriers were prepared and sterilised using the standard methods recommended by each of the manufacturers. Microcarriers prepared were: FACT and Pronectin F (Sigma, SoloHill) CGEN 102-L, HLX11-170, Pro-F 102-L, FACT 102-L, P102-L and P plus 102-L (Thermo Fisher). FACT and Pronectin F were used at 5 g/L, CGEN 102-L, HLX11-170, Pro-F 102-L, FACT 102-L, P102-L and P plus 102-L were used at 2.5 g/L. Shake flasks were seeded using 4×10⁵ AVDS2 cells per 250 ml flask in a 40 ml MEM cell culture growth medium MEM (Sigma M4655) supplemented with 10% fetal bovine serum (FBS). The flasks were placed in an orbital shaking incubator at 37° C. and 70 rpm. Flasks were harvested after 6 to 7 days and the viable cell concentration determined as described previously. The results are presented in Table 3.

TABLE 3
Growth of cell lines AVDS1, AVDS2 and AVDS3 on
Various Microcarriers

| | | | | | Harvest |
|---|---|---|---|---|---|
| | | Microcarrier | Time in | Harvest | viability |
| Cell | Microcarrier | Concentration | culture | cell | (% viable |
| line | type | (g/L) | (days) | number | cells) |
| AVDS2 | CGEN 102L | 2.5 | 6 | 3.8 × 10⁵ | 82 |
| AVDS1 | FACT | 5.0 | 7 | 2.7 × 10⁵ | 82 |
| AVDS2 | FACT | 5.0 | 6 | 5.0 × 10⁴ | 7 |
| AVDS2 | FACT 102L | 2.5 | 6 | 3.3 × 10⁵ | 55 |
| AVDS2 | HLX11-170 | 2.5 | 6 | 1.2 × 10⁶ | 89 |
| AVDS3 | HLX11-170 | 2.5 | 7 | 1.5 × 10⁶ | 93 |
| AVDS2 | P 102-L | 2.5 | 6 | 3.0 × 10⁴ | 18 |
| AVDS2 | P plus 102-L | 2.5 | 6 | 1.1 × 10⁵ | 46 |
| AVDS2 | ProF 102-L | 2.5 | 6 | 4.4 × 10⁵ | 57 |
| AVDS1 | Pronectin F | 5.0 | 7 | 3.0 × 10⁴ | 15 |
| AVDS2 | Pronectin F | 5.0 | 6 | 3.0 × 10⁴ | 26 |

The finding that only the HLX11-170 microcarriers supported any significant growth and proliferation (above the number of cells originally seeded (4×10⁵ cells)) of AVDS2 mesenchymal dermal stem cells was surprising. The HLX11-170 microcarriers are composed of polystyrene coated with cationic trimethyl ammonium. The other microcarriers which surprisingly did not support growth were composed of either a type I porcine collagen with a cationic charge, uncoated polystyrene plastic or plastic with either a surface charge or coated with recombinant fibronectin or fibronectin. The poor growth of AVDS2 cells was especially surprising given that the prior art demonstrates that a wide range of cells such as embryonic stem cell lines, bone marrow derived mesenchymal stem cell lines and lung fibroblast cell lines will readily grow on microcarriers composed of collagen.

### EXAMPLE 4

Eight 225 cm² cell culture flasks (Nunc) of dermal sheath cells AVDS4 grown in static culture conditions were detached and counted using methods well described in the art. 2.3×10⁷ AVDS4 cells were used to inoculate a glass cell culture bioreactor (Applikon) containing a total volume of 1.0 L MEM cell culture growth medium (Sigma M4655) supplemented with 10% fetal bovine serum (FBS), 0.2% Pluronic F-68 (Sigma P1300) and 5 g/L HLX11-170 microcarriers (which were prepared and sterilised as described previously). The bioreactor was cultured at a temperature of 36.5° C., pH 7.0 (manual control by carbon dioxide gas sparging and/or addition of sodium hydroxide) and an impeller speed of 80 rpm. After 2 days incubation under the conditions described a further 1.0 L MEM cell culture growth medium supplemented with 10% FBS and 0.2% pluronic F-68 was added aseptically to the cell culture bioreactor. The incubation was continued under the conditions described for a further five days. The bioreactor was then harvested and 4.4×10⁸ AVDS4 viable cells were recovered from the bioreactor. The results demonstrate that HLX11-170 microcarriers can be used for the reproducible generation of large numbers of stem cells with well-defined characteristics under tightly controlled and scaleable conditions.

### EXAMPLE 5

The AVDS4 cells recovered from the HLX11-170 microcarrier bioreactor culture described in Example 4 were used to prepare frozen cell stocks using methods well established in the art. After storage in liquid nitrogen for 4 days, 2 ampoules were thawed and diluted in 30 ml MEM cell culture growth medium (Sigma M4655) supplemented with 10% fetal bovine serum (FBS). This AVDS4 cell suspension was then used to seed 6 cell culture flasks as shown in Table 4. The shake flasks with HLX11-170 microcarriers were placed in an orbital shaking incubator at 37° C. and 70 rpm and the 225 cm² cell culture flasks were grown under static culture conditions (37° C., 5% carbon dioxide) with no microcarriers. Flasks were harvested after 5 to 6 days and the viable cell concentration determined as described previously.

TABLE 4
Growth of AVDS4 cells in static and shake flask culture
following harvest from HLX11-170 microcarriers

| | | | Total | Number of | Number of | Harvest | Harvest |
|---|---|---|---|---|---|---|---|
| Flask | | Microcarrier | volume | cells | days | cell | viability |
| Number | Flask type | concentration | (ml) | seeded | incubation | number | (%) |
| 1 | 225 cm² cell | No | 45 | 5.0 × 10⁵ | 5 | 2.1 × 10⁶ | 93 |
| | culture flask | microcarriers | | | | | |
| | (Nunc): static | | | | | | |
| | culture | | | | | | |
| 2 | 225 cm² cell | No | 45 | 7.5 × 10⁵ | 5 | 3.1 × 10⁶ | 94 |
| | culture flask | microcarriers | | | | | |
| | (Nunc): static | | | | | | |
| | culture | | | | | | |
| 3 | 225 cm² cell | No | 45 | 7.5 × 10⁵ | 5 | 3.2 × 10⁶ | 93 |
| | culture flask | microcarriers | | | | | |
| | (Nunc): static | | | | | | |
| | culture | | | | | | |
| 4 | 250 ml cell | 2.5 g/L | 50 | 5.0 × 10⁵ | 6 | 8.4 × 10⁴ | 88 |
| | culture shake | | | | | | |
| | flask (Corning) | | | | | | |
| 5 | 250 ml cell | 2.5 g/L | 50 | 7.5 × 10⁵ | 6 | 2.5 × 10⁵ | 84 |
| | culture shake | | | | | | |
| | flask (Corning) | | | | | | |
| 6 | 250 ml cell | 2.5 g/L | 50 | 1.0 × 10⁶ | 6 | 4.1 × 10⁵ | 87 |
| | culture shake | | | | | | |
| | flask (Corning) | | | | | | |

2.8×10⁶ AVDS4 cells from Flasks 2 and 3 (Table 4) were then used to seed a 1 L shake flask (Corning) in a total volume of 125 ml of MEM cell culture growth medium supplemented with 10% FBS with 5 g/L HLX11-170 microcarriers. The headspace was equilibrated with 5% CO₂ in air gas and the flask was transferred to an orbital shaker incubator at 37° C., 70 rpm. After 2 days incubation under the conditions described a further 125 mL MEM cell culture growth medium supplemented with 10% FBS was added to the flask. The incubation was continued under the conditions described for a further four days. The flasks was harvested and the viable cell concentration determined as described previously. No viable cells were recovered.

Surprisingly the HLX11-170 microcarriers (Flasks 4, 5 and 6) did not support growth of the AVSD4 cells (which had previously been harvested from a HLX11-170 microcarrier bioreactor culture—Example 5) and a lower number of cells were recovered from the flasks than originally seeded. The AVDS4 cells harvested from a HLX11-170 microcarrier bioreactor culture (Example 5) did grow and proliferate in static culture in the absence of microcarriers (Flasks 1, 2, 3).

Even more unexpected was the finding that AVDS4 cells harvested from Flasks 2 and 3 failed to attach and proliferate on HLX11-170 microcarriers.

The data suggest that although cells can be expanded using HLX11-170 microcarriers (Example 5) they do not attach and proliferate on HLX11-170 microcarriers if they have previously been harvested from HLX11-170 microcarrier culture.

### EXAMPLE 6

One 225 cm² cell culture flask (Nunc) and two 75 cm² cell culture flasks (Nunc) of dermal sheath cells AVDS5 grown in static culture were detached using methods well established in the art. 2.2×10⁶ and 1.1×10⁶ AVDS5 cells were then used to seed a 1 L cell culture shake flask (Nunc) and a 500 ml cell culture shake flask (Nunc) respectively. The cell culture flasks contained 1 g/L CultiSpher S microcarriers (prepared as described previously) in a total volume of 200 ml and 100 ml of MEM cell culture growth medium supplemented with 10% FBS in the 1 L and the 500 ml shake flasks respectively. The headspace was equilibrated with 5% CO₂ in air gas and the flasks were transferred to an orbital shaker incubator at 37° C., 40 rpm. After 7 days incubation under the conditions described, the contents of the flasks were harvested and cells were detached from the microcarriers. 9.1×10⁶ AVDS5 cells were used to inoculate a 2 L glass cell culture bioreactor (Applikon) in a total volume of 500 ml of MEM cell culture growth medium supplemented with 10% FBS+0.2% pluronic F-68 with 2 g/L CultiSpher S microcarriers. The bioreactor was cultured at 36.5° C., pH 7.0 (maintained as described in Example 6) and an agitator speed of 50 rpm. After 3 days incubation under the conditions described, 500 ml of the cell culture growth medium described above was added to the bioreactor and the agitator speed was increased to 60 rpm. After a total of 7 days incubation, the microcarriers were removed from the bioreactor and cells detached from the microcarriers. 1.3×10⁷ cells were harvested at a viability of 83%.

### EXAMPLE 7

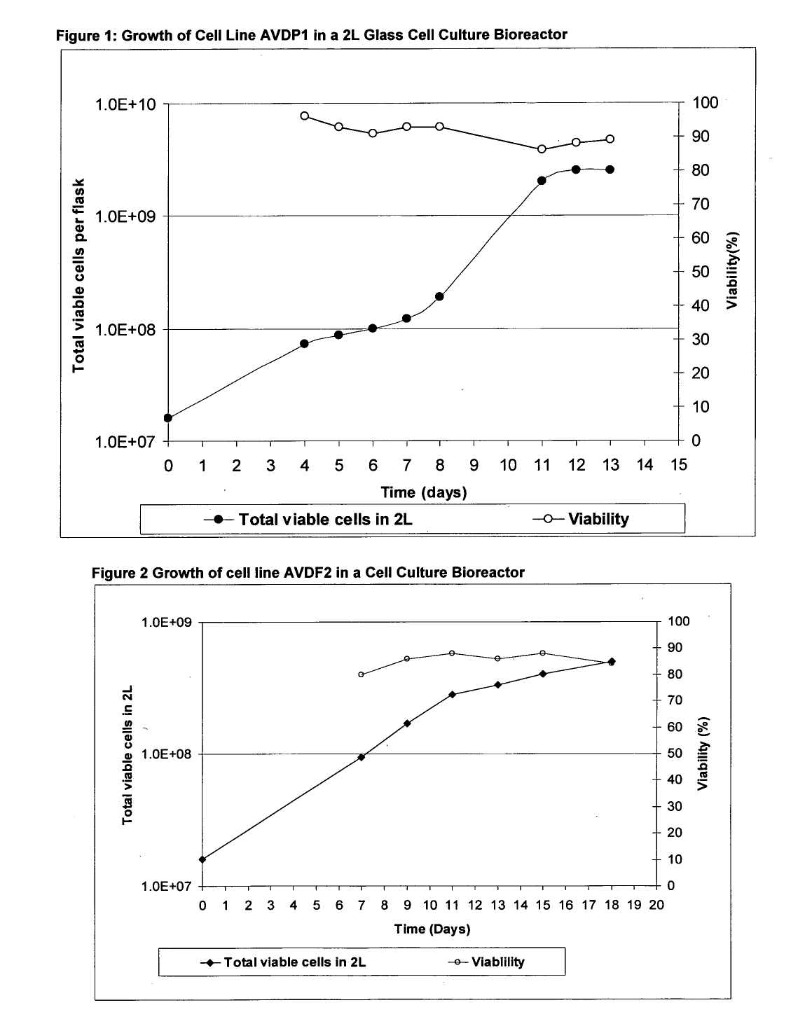

Three 225 cm² cell culture flasks (Nunc) of dermal papilla cells AVDP1 grown in static culture were detached. 2.0×10⁶ AVDP1 cells were used to seed each of two 1.5 L cell culture spinner flasks containing 1.5 g/L CultiSpher S microcarriers in a total volume of 250 ml of MEM cell culture growth medium supplemented with 10% FBS. The headspace was equilibrated with 5% CO₂ in air gas. The spinner flask was transferred to a cell culture incubator (37° C.) and agitated at 30 rpm using a magnetic stirrer base. After 4 days incubation under the conditions described, 125 ml spent cell culture growth medium was removed from each flask and replaced with fresh cell culture growth medium as described above. After 6 days incubation under the conditions described, the AVDP1 cells were detached from the microcarriers. 1.6×10⁷ cells were used to inoculate a 2 L glass cell culture bioreactor (Applikon) in a total volume of 2 L of MEM cell culture growth medium supplemented with 10% FBS, 0.2% pluronic F-68 and 1.5 g/L CultiSpher S. The bioreactor was cultured at 37° C., pH 7.10 (controlled as described in Example 6), dissolved oxygen tension 20% (air saturation) and an agitator speed of 50 rpm. The dissolved oxygen level was maintained using CO₂ and N₂ sparging. After 4 and 8 days incubation under the conditions described, 1 L of spent media was removed from the bioreactor and replaced with 1 L of fresh medium (as described above). After 4 days incubation, the agitator speed was increased to 70 rpm, and at 11 days incubation, the agitator speed was increased to 90 rpm. Samples were removed from the bioreactor periodically and the cells were detached and the number of viable cells determined as described previously. The viability and cell number are presented in FIG. 1. A significantly high peak viable cell density of 2.9×10⁹ AVDP1 cells was achieved.

The results of Examples 5, 6 and 7 demonstrate that gelatin microcarriers can also be used for the reproducible generation of significantly large numbers of stem cells with well-defined characteristics under tightly controlled and scaleable conditions.

### EXAMPLE 8

CultiSpher S microcarriers were prepared as described previously. The microcarriers were then used to culture dermal fibroblast cell line AVDF1. A 250 ml shake flask (Corning) with 25 ml MEM cell culture growth medium (Sigma M4655) supplemented with 10% fetal bovine serum (FBS) was seeded with 5×10⁵ AVDF1 cells and 1 g/L CultiSpher S microcarriers. The flask was placed in an orbital shaker incubator at 37° C. and 70 rpm. After 24 h incubation under the conditions described, a further 25 ml of the growth medium (as described above) was added to the flask and the flask returned to the shaker incubator and the incubation continued under the conditions described above. After 5 days incubation, the flask contents were harvested and the total concentration of viable cells determined as described previously. A total of 2.1×10⁶ viable cells were harvested.

### EXAMPLE 9

Two 225 cm² cell culture flasks (Nunc) of dermal fibroblast cells AVDF2 grown in static culture were detached and counted using methods well described in the art. 2.0×10⁶ AVDF2 cells were used to seed each of two 1 L cell culture shake flasks (Nunc) containing a total volume of 90 ml MEM cell culture growth medium supplemented with 10% FBS containing 2 g/L Cultispher S microcarriers (prepared as described previously). The headspace of the cell culture flasks was equilibrated with 5% CO₂ in air gas. The cell culture flasks were transferred to an orbital shaker incubator at a temperature of 37° C. and agitation at 40 rpm. The flasks were incubated under the conditions described for 24 h and a further 90 ml of cell culture growth medium (as described above) added to each flask. The flasks were returned to the shaker incubator under the conditions described above and the incubation continued for 7 days. The contents of the flasks were harvested and cells were detached (as described previously) from the microcarriers. 1.65×10⁷ AVDF2 cells were then used to inoculate a 3 L spinner cell culture flask (Corning) in a total volume of 750 ml MEM cell culture growth medium supplemented with 10% FBS containing 2 g/L CultiSpher S microcarriers. The headspace was equilibrated with 5% CO₂ in air gas. The spinner flask was transferred to a cell culture incubator (37° C.) and agitated at 30 rpm using a magnetic stirrer base. After 2 days incubation under the conditions described, a further 750 ml of the cell culture growth medium described above was added to the spinner flask. The incubation was continued under the conditions described above for a further 4 days. The contents of the cell culture spinner flask were harvested and cells recovered. 4.0×10⁷ AVDF2 cells were recovered at a viability of 91%.

### EXAMPLE 10

One 75 cm² flask (Nunc) of dermal fibroblast cells AVDF2 grown in static culture was detached and counted using methods well described in the art. 2.2×10⁶ AVDF2 cells were used to seed a 1 L spinner cell culture flask (Wheaton) in a total volume of 240 ml MEM cell culture growth medium supplemented with 10% FBS containing 1.5 g/L CultiSpher S microcarriers (prepared as previously described). The headspace of the flask was equilibrated with 5% CO₂ and 2% O₂ in nitrogen gas. The spinner flask was transferred to a cell culture incubator (37° C.) and agitated at 40 rpm using a magnetic stirrer base. After 4 days incubation under the conditions described, 120 ml of the cell culture supernatant was removed from the spinner flask and 120 ml of fresh cell culture growth medium (described above) was added to the spinner flask. The incubation was continued under the conditions described above for a further 24 hours. A further sample (90 ml) was taken from the cell culture spinner flask and cells recovered. 5.7×10⁶ AVDF2 cells were recovered at a viability of 76%. The recovered cells (2.4×10⁶ AVDF2 cells) were then used to seed each of two 1 L spinner cell culture flasks (Wheaton) in a total volume of 300 ml cell culture growth medium (described above) per flask containing 1.5 g/L

CultiSpher S microcarriers (prepared as previously described). The headspace of the flask was equilibrated with 5% CO₂ and 2% O₂ in nitrogen gas. The spinner flask was transferred to a cell culture incubator (37° C.) and agitated at 40 rpm using a magnetic stirrer base. After 4 days incubation under the conditions described, and on each day up to and including 7 days incubation, a volume of 30 ml of the cell culture supernatant was removed from each spinner flask and replaced with 30 ml fresh cell culture growth medium (described above). After a total of 8 days incubation under the conditions described, 6.6×10⁷ AVDF2 cells were recovered at a viability of 88%. These cells (1.6×10⁷ AVDF2 cells) were used to inoculate a 2 L glass cell culture bioreactor (Applikon) in a total volume of 2 L of MEM cell culture growth medium supplemented with 10% FBS, 0.2% pluronic F-68 and 1.5 g/L CultiSpher S microcarriers (prepared as described previously). The bioreactor was cultured at 37° C., pH 7.0 (controlled as described in Example 7), dissolved oxygen tension 2.0% (air saturation) and an agitator speed of 50 rpm which was increased gradually to 90 rpm over the course of the culture. The dissolved oxygen level in the cell culture was maintained using CO₂ and N₂ gas sparging. Emulsion C antifoam agent (Sigma) was added to the bioreactor when foaming occurred. After 4 days incubation under the conditions described, 20% of the total culture volume in the bioreactor was replaced with fresh cell culture growth medium every 24 hours. Samples were removed from the bioreactor periodically and the cells were detached and the number of viable cells determined as described previously. The viability and cell number are presented in FIG. 2. A viable cell number of 5.0×10⁸ cells was reached after 18 days in culture.

### EXAMPLE 11

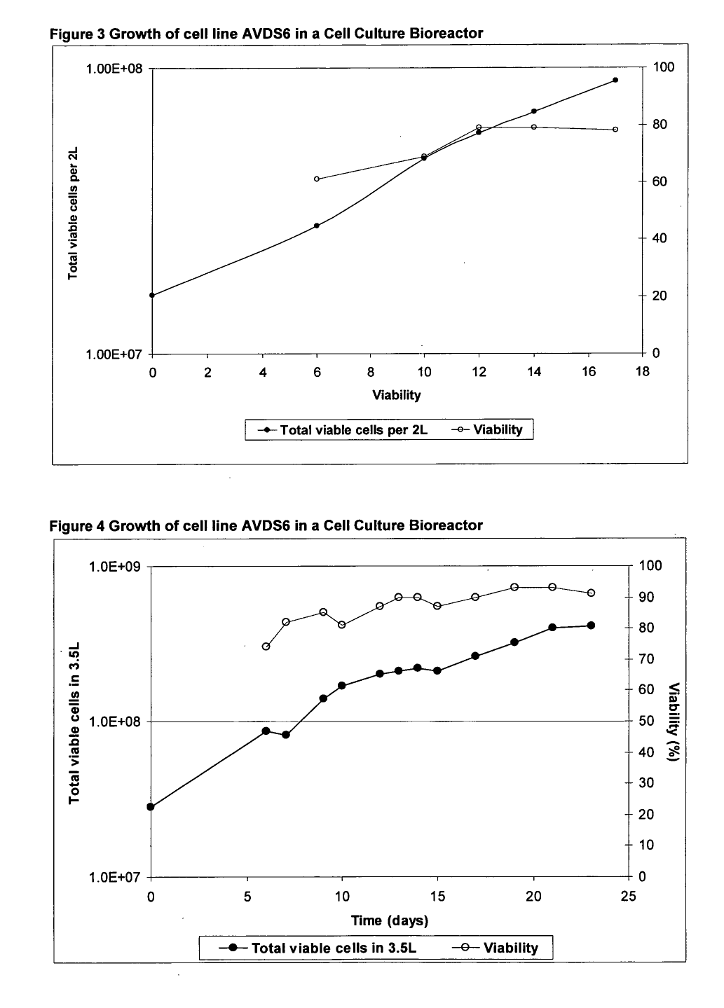

A vial of cell line AVDS6 was thawed and 50 ml of MEM cell culture growth medium supplemented with 10% FBS was added. 4.2×10⁶ cells at a viability of 90% were recovered. 2×10⁶ AVDS6 cells were used to seed each of two spinner cell culture flasks (Wheaton) with 8.0×10³ cells/mL and 1.5 g/L CultiSpher S microcarriers (prepared as described previously) in a total volume of 250 mL growth medium (described above). The flasks were incubated as described in Example 10. After 4, 5, 6 and 7 days incubation under the conditions described, a volume of 30 ml of the cell culture supernatant was removed from each spinner flask and replaced with 30 ml fresh cell culture growth medium (described above). After a total of 8 days incubation, the contents of the flasks were harvested and 4.9×10⁷ cells at a viability of 80% were recovered. 1.6×10⁷ AVDS6 cells were then used to inoculate a 2 L glass cell culture bioreactor (Applikon) in a total volume of 1.5 L of MEM cell culture growth medium supplemented with 10% FBS, 0.2% pluronic F-68 and 1.5 g/L CultiSpher S microcarriers (prepared as described previously). The bioreactor was cultured at 37° C., pH 7.0 (controlled as described in Example 7), dissolved oxygen tension 2.0% (air saturation) and an agitator speed of 50 rpm which was increased gradually to 90 rpm over the course of the culture. The dissolved oxygen level was maintained as described in Example 10. Emulsion C antifoam agent (Sigma) was added to the bioreactor when foaming occurred. After 4 days under the conditions described, a further 500 ml of growth medium (described above) was added to the bioreactor. From 5 days in culture, 5% of the total culture volume was replaced every 24 h. Samples were removed from the bioreactor periodically and the cells were detached and the number of viable cells determined (as described previously). The viability and cell number are presented in FIG. 3. A viable cell number of 9.0×10⁷ cells was observed in a 2 L bioreactor after 17 days in culture.

### EXAMPLE 12

The cells from five 225 cm² flasks (Nunc) of cell line AVDS6 grown in static culture were detached and counted using methods well described in the art. 2.0×10⁶ AVDS6 cells were used to seed each of two 1 L spinner cell culture flasks (Wheaton) in a total volume of 200 ml MEM cell culture growth medium supplemented with 10% FBS containing 1.5 g/L CultiSpher S microcarriers (prepared as described previously). The headspace was equilibrated with 5% CO₂ and 2% O₂ in nitrogen gas. The spinner flask was transferred to a cell culture incubator (37° C.) and agitated at 40 rpm using a magnetic stirrer base. After 4 days incubation under the conditions described, and on each day up to and including 6 days incubation, a volume of 20 ml of the cell culture supernatant was removed from each spinner flask and replaced with 20 ml fresh cell culture growth medium (described above). After 7 days incubation under the conditions described, the flasks were harvested and 3.6×10⁷ cells at a viability of 87% were recovered. 2.8×10⁷ AVDS6 cells were then used to inoculate a 5 L glass cell culture bioreactor (Applikon) using a total volume of 3.5 L of MEM cell culture growth medium supplemented with 10% FBS, 0.2% pluronic F-68 and 1.5 g/L CultiSpher S microcarriers (prepared as described previously). The bioreactor was cultured at a temperature of 36.5° C., pH at 7.1 (controlled as described in Example 7), dissolved oxygen tension 5.0% (air saturation) and an agitator speed of 70 rpm (which was increased gradually to 110 rpm over the course of the culture). The dissolved oxygen level was maintained as described in Example 10. Emulsion C antifoam agent (Sigma) was added to the bioreactor when foaming occurred. From 4 to 7 days incubation under the conditions described, 10% of the total culture volume was replaced every 24 hours. From 8 to 10 days in culture, 15% of the total culture medium and from 11 to 22 days in culture, 20% of the total culture volume was replaced every 24 hours. Samples were removed from the bioreactor periodically and the cells were detached and the number of viable cells determined (as described previously). The viability and cell number are presented in FIG. 4. A maximum viable cell number of 4.1×10⁶ cells was observed in a 5 L bioreactor after 23 days in culture, demonstrating successful process scale up to 5 L bioreactor scale.

### EXAMPLE 13

Three 225 cm² flasks (Nunc) of cell line AVDS6 grown in static culture were detached and counted using methods well described in the art. 4.3×10⁶ cells at a viability of 84% were recovered. 3.0×10⁶ AVDS6 cells were then used to seed a 1 L spinner cell culture flask (Wheaton) in a total volume of 300 ml low serum (2%) growth medium supplemented with 4 mM glutamine (Sigma) containing 1.5 g/L CultiSpher S microcarriers (prepared as described previously). The culture was incubated as described in Example 12. After 6 days incubation under the conditions described, the contents of the flask was harvested and a total of 3.8×10⁷ cells at a viability of 93% were recovered. 2.0×10⁷ AVDS6 cells were used to inoculate a glass cell culture bioreactor (Applikon) using a total volume of 2.0 L of serum free growth medium supplemented with 4 mM glutamine (Sigma) 0.2% pluronic F-68 and 1.5 g/L CultiSpher S microcarriers (prepared as described previously). The bioreactor was cultured at a temperature of 36.5° C. and pH 7.1 (controlled as described in Example 7), dissolved oxygen tension 5.0% (air saturation) and an agitator speed of 70 rpm which was increased gradually to 130 rpm over the course of the culture. The dissolved oxygen level was maintained as described in Example 10. Emulsion C antifoam agent (Sigma) was added to the bioreactor when foaming occurred. From 4 to 7 days incubation under the conditions described, 10% of the total culture volume was replaced with fresh growth medium every 24 h. From 8 to 10 days in culture, 15% of the total culture volume and from 11 to 20 days in culture, 20% of the total culture volume was replaced with fresh growth medium every 24 h. Samples were removed from the bioreactor periodically and the cells were detached and the number of viable cells determined as described previously. The viability and cell number are presented in FIG. 5. A maximum viable cell number of 1.2×10⁸ cells was observed after 20 days in culture. This demonstrates the broad utility of the process of the present invention. In addition, those familiar with the art will recognise the benefits of growing cells using low serum medium.

### EXAMPLE 14

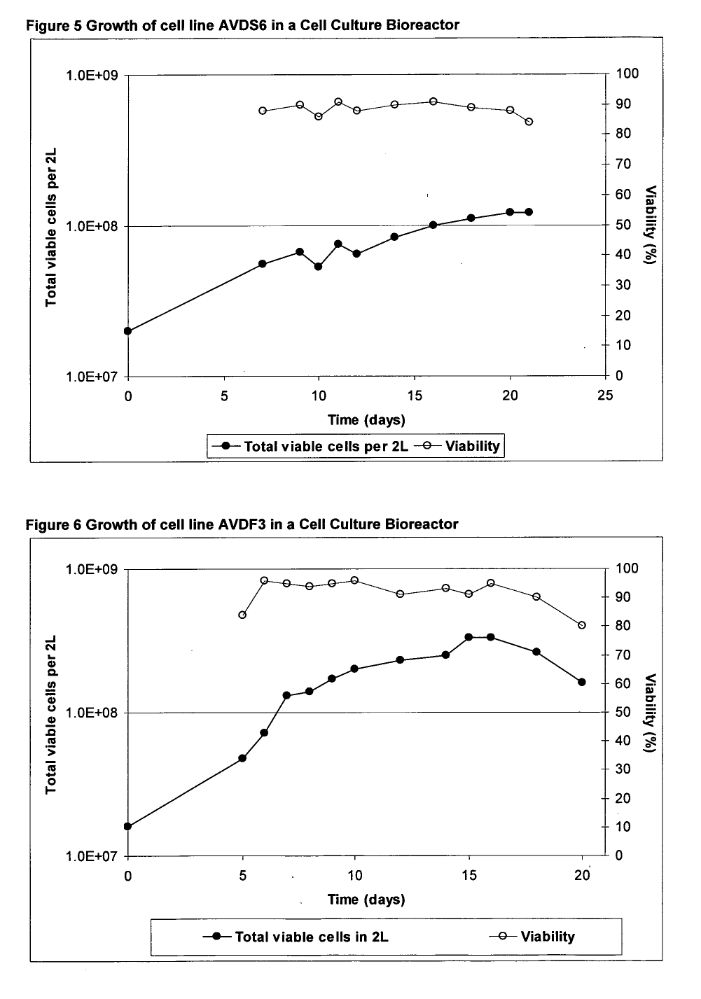

A vial of cell line AVDF3 was thawed and re-suspended using 45 ml of MEM cell culture growth medium supplemented with 10% FBS. 7.7×10⁶ AVDF3 cells at a viability of 94% were recovered. 2.4×10⁶ cells were used to seed each of two 1 L spinner cell culture flasks (Wheaton) with 8.0×10³ cells/mL and 1.5 g/L CultiSpher S microcarriers (prepared as described previously) in a total volume of 300 mL growth medium (described above). The flasks were incubated as described in Example 10. After 8 days incubation under these conditions the flasks were harvested and 8.4×10⁷ cells at a viability of 96% were recovered. 1.6×10⁷ cells were used to inoculate a glass cell culture bioreactor (Applikon) in a total volume of 2 L of MEM cell culture growth medium supplemented with 10% FBS, 0.2% pluronic F-68 and 1.5 g/L CultiSpher S microcarriers (prepared as described previously). The bioreactor was cultured at a temperature of 36.5° C., pH 7.0 (controlled as described in Example 7), dissolved oxygen tension 5.0% (air saturation) and an agitator speed of 70 rpm which was increased gradually to 120 rpm over the course of the culture. The dissolved oxygen level was maintained as described in Example 10. Emulsion C antifoam agent (Sigma) was added to the bioreactor when foaming occurred. From 4 to 7 days under the conditions described, 10% of the total culture volume was replaced with fresh growth medium every 24 h. From 8 to 10 days in culture, 15% of the total culture volume and from 11 to 20 days in culture, 20% of the total culture volume was replaced with fresh growth medium every 24 hours. Samples were removed from the bioreactor periodically and the cells were detached and the number of viable cells determined as described previously. The viability and cell number are presented in FIG. 6. A maximum viable cell number of 3.3×10⁸ cells was achieved on day 15.

### EXAMPLE 15

One 225 cm2 cell culture flask (Nunc) of dermal fibroblast cells AVDF3 grown in static culture conditions were detached and counted using methods well described in the art. 2.3×10⁶ cells were used to seed a 1.5 L cell culture spinner flask containing 1.5 g/L CultiSpher S microcarriers (prepared as described previously) in a total volume of 330 ml of serum free growth medium supplemented with 2 mM glutamine (Sigma). The headspace of the flask was equilibrated with 5% CO₂, 2% O₂ gas. The spinner flask was transferred to a cell culture incubator at 37° C. and agitated at 35 rpm using a magnetic stirrer base. After 4 days incubation under the conditions described 35 ml of cell culture supernatant was removed from the flask and replaced with fresh serum free growth medium (as described above). On day 5 and day 7, 50 ml of culture supernatant was removed and replaced with fresh serum free medium as described above. On day 8, 80 ml of culture supernatant was removed and replaced with fresh serum free medium. After 10 days in culture the AVDF3 cells were detached from the microcarriers. 1.35×10⁷ cells were used to inoculate a glass cell culture bioreactor (Applikon) in a total volume of 2 L of serum free growth medium supplemented with 2 mM glutamine, 0.2% pluronic F-68 and 1.5 g/L Cultispher S microcarriers (prepared as described previously). The bioreactor was cultured at a temperature of 36.5° C., pH 7.0 (controlled as described in Example 7), dissolved oxygen tension 5.0% (air saturation) and an agitator speed of 40 rpm which was increased gradually to 60 rpm over the course of the culture. The dissolved oxygen tension was maintained as described in Example 10. Emulsion C antifoam agent (Sigma) was added to the bioreactor when foaming occurred. After 4 days incubation under the conditions described, 200 ml of culture supernatant was removed from the bioreactor and replaced with fresh serum free growth medium (as described above). A further 200 ml of culture supernatant was removed and replaced with fresh serum free growth medium (as described above) on days 6, 8, 10, 11, 13, 15, 17 and 21. Samples were removed from the bioreactor periodically, the cells detached from the microcarriers and the number of viable cells determined as described previously. A peak cell density of 7.3×10⁷ cells was achieved on Day 15. This further demonstrates the utility of the process. The benefits of using serum free medium will be evident to those skilled in the art, in particular, reproducibility of production, more consistent performance, decrease in possibility contamination of the cells with adventitious agents. Serum free media are known in the art and are readily available from commercial suppliers.

### EXAMPLE 16

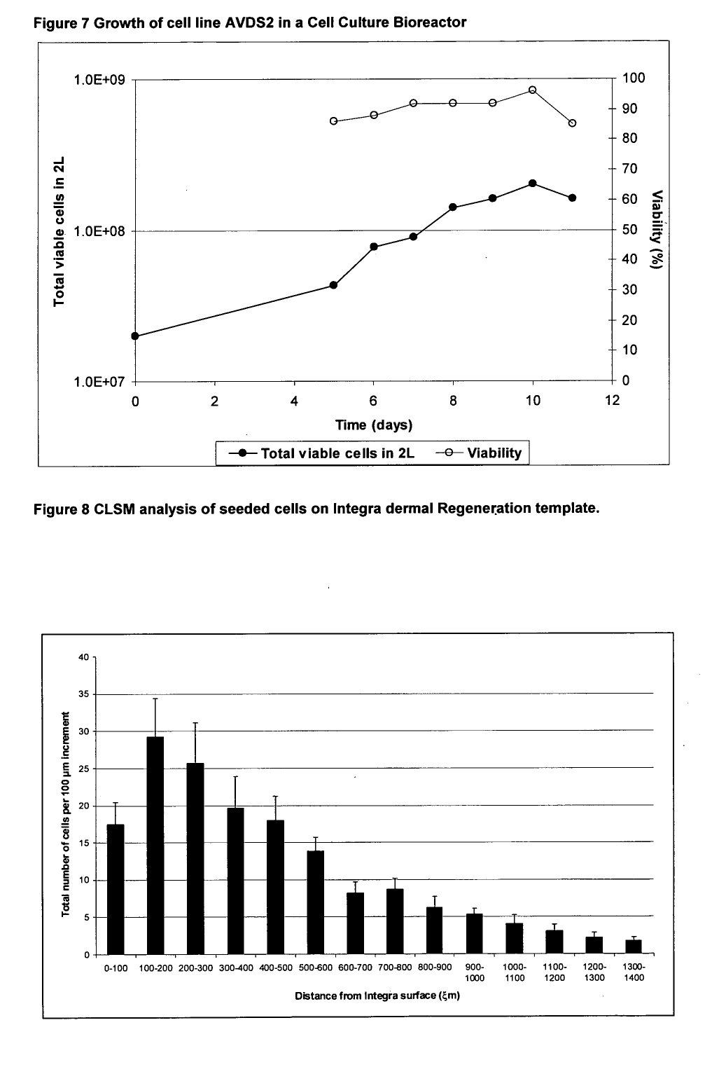

The cells from two 225 cm² flasks (Nunc) of cell line AVDS2 grown in static culture conditions were detached and counted using methods well described in the art. 3.1×10⁶ cells at a viability of 90% were recovered. 2.9×10⁶ AVDS2 cells were used to seed each of two 1 L spinner cell culture flasks (Wheaton) in a total volume of 300 ml MEM cell culture growth medium supplemented with 10% FBS containing 1.5 g/L CultiSpher S microcarriers (prepared as described previously). The headspace of the flask was equilibrated with 5% CO₂ and 2% O₂ in nitrogen gas. The spinner flask was transferred to a cell culture incubator (37° C.) and agitated at 40 rpm using a magnetic stirrer base. After 4 days incubation under the conditions described, and on each day up to and including 7 days incubation, a volume of 30 ml of the cell culture supernatant was removed from each spinner flask and replaced with 30 ml fresh cell culture growth medium (described above). After 8 days incubation under the conditions described, the flasks were harvested and 4.7×10⁷ cells at a viability of 96% were recovered. 2.0×10⁷ AVDS2 cells were used to inoculate a glass cell culture bioreactor (Applikon) in a total volume of 2 L of MEM cell culture growth medium supplemented with 10% FBS, 0.2% pluronic F-68 and 1.5 g/L CultiSpher S (prepared as described previously). The bioreactor was cultured at a temperature of 36.5° C., pH 7.1 (controlled as described in Example 7), dissolved oxygen tension 5.0% (air saturation) and an agitator speed of 40 rpm which was increased gradually to 70 rpm over the course of the culture. The dissolved oxygen level was maintained as described in Example 10. Emulsion C antifoam agent (Sigma) was added to the bioreactor when foaming occurred. From 4 to 7 days under the conditions described, 10% of the total culture medium was replaced every 24 hours and from 8 to 10 days in culture 15% of the total culture medium was replaced every 24 hours. The impeller (agitation) configuration used in this example was altered to that in Examples 7 to 15. The impeller configuration used in this example consisted of 2 impellers with a ratio of impeller diameter to bioreactor diameter of 0.4 and 0.3. In Examples 7 to 15, an impeller of a ratio of 0.3 was used at the base of the impeller shaft, i.e. located at the base of the cell culture vessel. In this example, the impeller with a 0.4 diameter ratio was used at the base of the impeller shaft/base of the cell culture vessel, and the impeller with a 0.3 diameter ratio was attached to the impeller shaft in a central position (relative to the impeller at the base of the shaft and the final cell culture medium volume (at harvest). Samples were removed from the bioreactor periodically and the cells were detached and the number of viable cells determined as described previously. The viability and cell number are presented in FIG. 7. A maximum viable cell number of 2.0×10⁸ cells was observed after 10 days in culture.

Surprisingly, the altered impeller configuration lead to a significant improvement to the process. A lower agitation speed could be used during the cell culture—reducing the potential negative effects of shear stress on the cells without influencing the optimum mixing required to maintain good cell growth and viability. More significantly, the cell culture time taken to reach a high viable cell number was significantly reduced. In Example 13, it can be seen that a peak cell number for a DS cell line was reached on day 23. In the present example, a similar peak cell number was reached on day 11 reducing cell culture time significantly. It will be evident to those skilled in the art how a reduction in cell manufacturing time will reduce manufacturing time and costs when the process is scaled-up to manufacture cells commercially for therapeutic applications.

### EXAMPLE 17

Skin-derived precursors (SKPs) are a self-renewing, multipotent precursor population of cells which can be isolated from dermal cells. SKPs cells have been well described in the art as exhibiting a similar gene expression profile to embryonic neural crest stem cells and thus can be used to generate neural crest derivatives, including Schwann cells and neurons of the peripheral nervous system. SKPs cells can be isolated by culturing dermal cells using high concentrations of fibroblast growth factor 2 (FGF2) and epidermal growth factor (EGF) in the growth medium. The SKPs proliferate as floating spheres and thus are easily visualised and quantified using methods well established in the art. Cells generated in the examples described above were used in a SKPs formation assay after growth under conditions described in the previous examples. The ability of cells to form SKPs after expansion/growth using bioreactor conditions was used as one measure of multipotency and thus confirm that the therapeutic potential of these cells had not be adversely influenced following bioreactor expansion/cell product manufacturing.

A vial of cell line AVDS5 cryopreserved from the cells harvested from the bioreactor culture (Example 6) was thawed and 44 ml MEM cell culture growth medium supplemented with 10% FBS was added. The cell suspension was transferred to a 225 cm² cell culture flask (Nunc) and incubated under static culture conditions described above for 6 days. The cells were then detached and counted and a total of 8.3×10⁵ cells at a viability of 80% were recovered. The cells were then centrifuged at 180×g for 5 minutes and resuspended in SKPs growth medium consisting of 74% DMEM (Sigma), 24% F12 with Glutamax (Invitrogen), 0.1% Penicillin/Streptomycin (Sigma), 40 ng/mL human FGF-2 (R&D Systems) 20 ng/mL human EGF (R&D Systems) and 2% B27 supplement (Invitrogen). 2.5×10⁵ cells were then transferred to a 25 cm² cell culture flask (Nunc) and incubated as described above. Every 3 to 4 days, 1 ml of cell-free supernatant was removed from the flask and replaced with 1 ml of SKPs growth medium containing a 5 times greater concentration of EGF, FGF-2 and B27 supplement.

After 22 days incubation, SKPs were visible in the flask indicating that the bioreactor expansion using CultiSpher S microcarriers described in Example 6 had not adversely influenced the cells in their ability to form SKPs and confirmed their multipotent stem cell characteristics. Those skilled in the art will appreciate the importance of the retention of cell properties on expansion particularly, though not limited to, retention of stem cell multipotency.

A vial of cell line AVDS4 cryopreserved from a stock of cells that had not been grown in a bioreactor (passaged using static culture in cell culture flasks only) was thawed and 15 ml MEM cell culture growth medium supplemented with 10% FBS was added. The cell suspension was transferred to two 25 cm² cell culture flasks (Nunc) and incubated under the conditions described above for 1 day. The cells were then detached from the flasks and used to seed a 225 cm² cell culture flask (Nunc) in a total volume of 45 ml of cell culture growth medium. After 4 days incubation under the conditions described, the cells were detached and 1.3×10⁶ cells at a viability of 88% were recovered. The cells were then centrifuged at 180×g for 5 minutes and resuspended in SKPs growth medium as described above. 2.5×10⁵ cells were then transferred to a 25 cm² cell culture flask (Nunc) and incubated as described above. Every 3 to 4 days, 1 ml of cell supernatant was removed from the flask and replaced with 1 ml of SKPs growth medium containing a 5 times greater concentration of EGF, FGF-2 and B27 supplement.

After 22 days incubation, SKPs were visible in the flask.

This example demonstrates that cells grown on CultiSpher S microcarriers retained their ability to form SKPs confirming that the cells had retained the capability vs. multipotent stem cell characteristics. This was particularly surprising since cells grown on HLX11-170 microcarriers would not form SKPs, and therefore had lost this key multipotency potential.

### EXAMPLE 18

Two 225 cm² cell culture flasks (Nunc) of cell line AVDS4 was harvested and 4.7×10⁶ cells at a viability of 91% were recovered. 2×10⁶ cells were then used to seed a 1 L spinner cell culture flask (Wheaton) in a total volume of 250 ml MEM cell culture growth medium supplemented with 10% FBS containing 1.5 g/L CultiSpher S microcarriers (prepared as described previously). The headspace of the flask was equilibrated with 5% CO₂ and 2% O₂ in nitrogen gas. The spinner flask was transferred to a cell culture incubator (37° C.) and agitated at 40 rpm using a magnetic stirrer base. After 4 days incubation under the conditions described, and on each day up to and including 6 days incubation, a volume of 25 ml of the cell culture supernatant was removed from each spinner flask and replaced with 25 ml fresh cell culture growth medium (described above). After 7 days incubation under the conditions described, the flask was harvested and 2.1×10⁷ cells at a viability of 95% were recovered. 1.3×10⁵ cells were then seeded into a 125 ml cell culture shake flask (Corning) containing a 12.5 cm² piece of a 3-dimensional biocompatible tissue regeneration scaffold (Integra Dermal Regeneration template (Integra Life Sciences)) in a total volume of 17 ml cell culture growth medium (described above). The Integra Dermal Regeneration template was positioned in the flask so that the silicon layer faced the cell culture flask base and the collagen surface was faced upwards and thus bathed in cell suspension/cell growth medium. The headspace of the flask was equilibrated with 5% CO₂ and 2% O₂ in nitrogen gas and transferred to an orbital shaking incubator set to 37° C. and 60 rpm. Each 3 to 4 days, 8 ml of cell supernatant was removed from the flask and replaced with 8 ml of fresh cell culture growth medium. The flask was incubated under these conditions for 14 days and then the Integra Dermal Regeneration template seed with cells was removed from the flask and fixed with a 50/50 mix of methanol/acetone (Fisher) using methods well described in the art. The infiltration and proliferation of cells into the Integra Dermal Regeneration template was then analysed by staining cell nuclei with propidium iodide (10 μg/ml, excited at 543 nm with emission captured in the range 610-640 nm), bisection and confocal laser scanning microscopy (CLSM) as is established in the art. Captured CLSM images were analysed using image analysis software (Image J, NIH). CLSM scans of cross sections of the Integra Dermal Regeneration template were analysed and the cell numbers (spatial distribution from the surface into the scaffold) are presented in FIG. 8.

The data indicate infiltration and proliferation of the cells into the Integra Dermal Regeneration template. These data further exemplify the utility of the process demonstrating that cells manufactured using CultiSpher S microcarrier culture retain the ability to infiltrate and proliferate within a biocompatible 3-dimensional scaffold such as Integra Dermal Regeneration template. It will be evident to those skilled in the art of dermal regeneration that if the cells are present only on the outer surface of a scaffold then critical aspects of wound healing such as, but not limited to, extracellular matrix production and vascularisation will be limited and the likelihood of successful tissue regeneration would below/poor.

### EXAMPLE 19

It is well established in the art that apoptosis is an important and active regulatory pathway of cell growth and proliferation. Apoptosis may be effected by (but limited to) growth media, stress conditions or other parameters. Determination of the level of apoptotic cells in a cell culture population can be used as a tool to monitor and assess the negative effects of process conditions, cell expansion, etc. The annexin V assay is well established in the art and was used to measure apoptosis in cells grown on CultiSpher S microcarriers in a bioreactor with MEM+10% FBS cell culture growth medium. Cells from continued passage in static culture, also grown in MEM+10% FBS, were used as a control to compare to the level of apoptosis in cell populations grown using CultiSpher S microcarriers in bioreactors.

A vial of cell line AVDS4 cryopreserved from a stock of cells that had been harvested from a bioreactor (Example 11) was thawed and seeded into a 225 cm² flask in MEM+10% FBS. Control culture was produced by expanding AVDS4 from a cell bank seeded at the same level as the flask described previously into a 225 cm² flask in MEM+10% FBS. This control culture had not been processed using microcarriers or bioreactor culture.

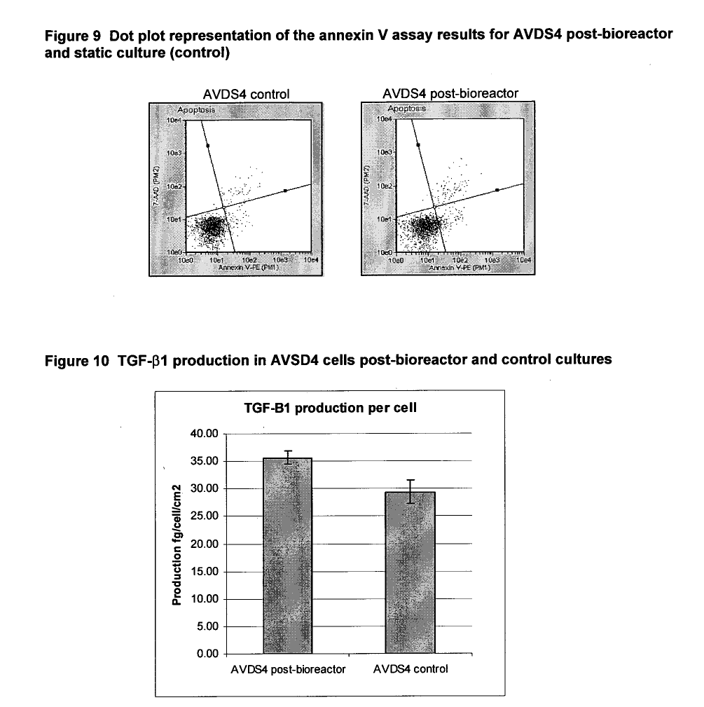

Both flasks were incubated for 5 days at 37° C., 5% CO₂ and then each cell culture was prepared for analysis as follows. The growth medium was separated from the cell monolayer. The cell monolayer was washed and then detached from the tissue culture flask as is well established in the art. The growth medium, wash and detached monolayer were then re-combined. The cells were centrifuged at 300×g for 5 minutes at room temperature. The supernatant was aspirated and the cell pellet was resuspended in fresh MEM+10% FBS at 5×10⁵ cells/ml. 100 μl of this cell suspension was transferred to a microcentrifuge tube containing 100 μl of Guava Nexin Reagent (Guava Technologies Inc, Hayward, USA). After mixing the cells/reagent were incubated at room temperature in the dark for 20 minutes. The sample was then analysed using a Guava PCA cytometer (according to the Guava PCA manufacturers guide). Both cultures achieved similar viable cell counts after 5 days incubation. A dot plot representation of assay results are shown in FIG. 9 and the level of apoptotic cells in each population is presented in Table 5.

TABLE 5
Apoptosis measurements in cell line AVDS4 post-bioreactor
and static culture (control)

| | | Early | Late |
|---|---|---|---|
| | Viable cells | apoptotic cells | apoptotic/dead |
| Culture | (%) | (%) | cells (%) |
| AVDS4 control | 94 | 3 | 3 |
| AVDS4 post-bioreactor | 89 | 6 | 5 |

Remarkably, cells have been cultured in a bioreactor using CultiSpher microcarriers and that were in culture for a significantly longer period than the control cells, show comparable and acceptable cell viability/level of apoptotic cells.

### EXAMPLE 20

Transforming growth factor beta 1 (TGF-β1) is a polypeptide member of the transforming growth factor beta superfamily of cytokines. It is a secreted protein that performs many cellular functions and has been identified as having a key role in wound healing. A TGF-β1 duo ELISA assay (R&D Systems) was used to determine levels of the protein in culture supernatant from cells grown on CultiSpher S microcarriers in a bioreactor with MEM+10% FBS cell culture growth medium. Cells from continued passage in static culture using tissue culture flasks, also grown in MEM+10% FBS, were used as a control to compare to the levels of TGF-β1 produced by the two cell populations.

A vial of cell line AVDS4 cryopreserved from a stock of cells that had been harvested from a bioreactor (Example 11) was thawed and seeded into a 225 cm² flask in MEM+10% FBS. Control culture was produced by expanding AVDS4 from a cell bank seeded at the same level as the flask described previously into a 225 cm² flask in MEM+10% FBS.

This control culture had not been processed using microcarriers or bioreactor culture. Both flasks were incubated for 5 days at 37° C., 5% CO₂ and the residual cell culture medium was then harvested and assayed to determine the concentration of TGF-β1 using a ELISA assay (R&D Systems). Samples were analysed in duplicate and the results are shown in FIG. 10. The TGF-β1 levels were normalised to cell concentration vs. flask surface area.

The results clearly demonstrate that cells expanded using microcarriers in bioreactor culture continue to express TGF-β1 at a level equivalent to cells that have been passaged using static culture and thus have maintained this key quality attribute.

1-10. (canceled)

11. A process for cultivation of differentiated human cells retaining stem cell potential which comprises culturing differentiated human cells retaining stem cell potential anchored to a microcarrier selected from the group consisting of gelatin microcarriers and quaternary ammonium derivatised polystyrene microcarriers.

12. A process according to claim 11, wherein the cells are adult cells.

13. A process according to claim 12, wherein the cells are mesenchymal cells.

14. A process according to claim 13, wherein the cells are dermal cells.

15. A process according to claim 14, wherein the cells are dermal sheath cells, dermal fibroblast or dermal papilla cells.

16. A process according to claim 11, wherein the cultivation process comprises cultivation of cells, harvesting said cells and reattachment of cells to a microcarrier for further cultivation.

17. A process according to claim 16, wherein a gelatin microcarrier is employed through the cultivation process.

18. A process according to claim 16, wherein a quaternary ammonium derivatised polystyrene microcarrier is employed as initial or final microcarrier.

19. A process according to claim 11 which is operated using fed batch or continuous cell culture conditions.

20. A process according to claim 11, wherein the culture vessel is agitated with two or more impellers mounted about a common shaft, at least one impeller being located in the lower third of the medium in the culture vessel, and at least one impeller located in the middle third of the medium in the vessel or in the top third of medium in the vessel.

21. A process according to claim 20, wherein each impeller comprises two, three or four blades angled compared with the axis of the impeller shaft(s).

22. A process according to claim 20, wherein the impellers have a diameter:vessel diameter ratio of at least 0.25:1.

23. A process according to claim 20, wherein the impellers have a diameter:vessel diameter ratio in the range from 0.3:1 to 0.7:1.

24. A process according to claim 20, wherein a gelatin microcarrier is employed through the cultivation process.

25. A process according to claim 20, wherein a quaternary ammonium derivatised polystyrene microcarrier is employed as initial or final microcarrier.

26. A process according to either claim 24 or 25, wherein the cultivation process comprises cultivation of cells, harvesting said cells and reattachment of cells to a microcarrier for further cultivation.

27. A process according to claim 26, wherein the cells are dermal sheath cells, dermal fibroblast or dermal papilla cells.

28. A process according to claim 27, wherein the impellers have a diameter:vessel diameter ratio in the range from 0.3:1 to 0.7:1.
